(19) United States
(12) Patent Application Publication
(10) Pub. No.: US 2007/0073098 A1
(43) Pub. Date: Mar. 29, 2007

(54) Method and apparatus for adjusting body lumens

(75) Inventors: Jay A. Lenker, Laguna Beach, CA (US); George F. Kick, Casa Grande, AZ (US); Samuel Shaolian, Newport Beach, CA (US); Shawn Moaddeb, Irvine, CA (US); Mike Henson, Coto De Caza, CA (US)

Correspondence Address: MCDERMOTT WILL & EMERY LLP, 18191 VON KARMAN AVE., SUITE 500, IRVINE, CA, 92612-7108, US

(73) Assignee: Ellipse Technologies, Inc., Irvine, CA (US)

(21) Appl. No.: 11/525,480

(22) Filed: Sep. 22, 2006

Related U.S. Application Data
(60) Provisional application No. 60/720,136, filed on Sep. 23, 2005.

(51) Int. Cl. A61F 2/02

(52) U.S. Cl. 600/30

(57) ABSTRACT

Disclosed is a device and method for accessing the lower esophageal sphincter through the esophagus. In one embodiment, a catheter is inserted through the mouth or nose of a patient and advanced to the region of the diaphragm. Under fluoroscopy or endoscopy, a hollow needle at the distal end of the catheter punctures the wall of the esophagus from the inside so that the distal end of the needle is positioned outside the esophagus. An implant is next advanced out through the hollow needle to the region outside the sphincter where it is deflected and coerced to bluntly dissect around the circumference of the esophagus, where the implant is left in place to heal. The hollow needle is removed and the esophageal wall is allowed to heal. Subsequent diametric adjustment of the implant allows for tightening or loosening of the sphincter to minimize gastric reflux. The device and method can also be used to treat the pyloric or other body sphincters, hollow organs, or ducts.

### PRIORITY CLAIM

This application claims the benefit of U.S. Provisional Application Ser. No. 60/720136, filed on Sep. 23, 2005, and titled METHOD AND APPARATUS FOR ADJUSTING SPHINCTER FUNCTION, the entirety of which is hereby incorporated by reference.

### BACKGROUND OF THE INVENTION

1. Field of the Invention

The invention relates to medical devices for transluminally accessing and controlling a diameter of body lumens and cavities along a mammalian alimentary canal, including methods and devices for performing diagnosis and therapeutic intervention to reduce obesity and to correct gastro esophageal reflux disease.

2. Description of the Related Art

The lower esophageal sphincter (LES) is a ring of increased thickness in the circular, smooth muscle layer of the esophagus. At rest, the lower esophageal sphincter maintains a high-pressure zone between 15 and 30 mm Hg above intragastric pressures. The lower esophageal sphincter relaxes before the esophagus contracts, and allows food to pass through to the stomach. After food passes into the stomach, the sphincter constricts to prevent the contents from regurgitating into the esophagus. The resting tone of the LES is maintained by myogenic (muscular) and neurogenic (nerve) mechanisms. The release of acetylcholine by nerves maintains or increases lower esophageal sphincter tone. It is also affected by different reflex mechanisms, physiological alterations, and ingested substances. The release of nitric oxide by nerves relaxes the lower esophageal sphincter in response to swallowing, although transient lower esophageal sphincter relaxations may also manifest independently of swallowing. This relaxation is often associated with transient gastro esophageal reflux in normal people.

Gastro esophageal reflux disease, commonly known as GERD, results from incompetence of the lower esophageal sphincter, located just above the stomach in the lower part of the esophagus. Acidic stomach fluids may flow retrograde across the incompetent lower esophageal sphincter into the esophagus. The esophagus, unlike the stomach, is not capable of handling highly acidic contents so the condition results in the symptoms of heartburn, chest pain, cough, difficulty swallowing, or regurgitation. These episodes can ultimately lead to injury of the esophagus, oral cavity, the trachea, and other pulmonary structures. GERD affects a large proportion of the population and mild cases can be treated with lifestyle modifications and pharmaceutical therapy. Patients, who are resistant, or refractory, to pharmaceutical therapy or lifestyle changes are candidates for surgical repair of the lower esophageal sphincter. The most common surgical repair, called fundoplication surgery, generally involves manipulating the diaphragm, wrapping the upper portion of the stomach, the fundus, around the lower esophageal sphincter, thus tightening the sphincter, and reducing the circumference of the sphincter so as to eliminate the incompetence. The hiatus, or opening in the diaphragm is reduced in size and secured with 2 to 3 sutures to prevent the fundoplication from migrating into the chest cavity. The repair can be attempted through open surgery, laparoscopic surgery, or an endoscopic, or endoluminal, approach by way of the throat and the esophagus. The open surgical repair procedure, most commonly a Nissen fundoplication, is effective but entails a substantial insult to the abdominal tissues, a risk of anesthesia-related iatrogenic injury, a 7 to 10 day hospital stay, and a 6 to 12 week recovery time, at home. The open surgical procedure is performed through a large incision in the middle of the abdomen, extending from just below the ribs to the umbilicus (belly button).

Very recently, endoscopic techniques for the treatment of GERD have been developed. Laparoscopic repair of GERD has the promise of a high success rate, currently 90% or greater, and a relatively short recovery period due to minimal tissue trauma. Laparoscopic Nissen fundoplication procedures have reduced the hospital stay to an average of 3 days with a 3-week recovery period at home. Another type of laparoscopic procedure involves the application of radio-frequency waves to the lower part of the esophagus just above the sphincter. The waves cause damage to the tissue beneath the esophageal lining and a scar (fibrosis) forms. The scar shrinks and pulling on the surrounding tissue, thereby tightening the sphincter and the area above it. These radio-frequency waves can also be used to create a controlled neurogenic defect, which may negate inappropriate relaxation of the LES. A third type of endoscopic treatment involves the injection of material or devices into the esophageal wall in the area of the lower esophageal sphincter. This increases the pressure in the lower esophageal sphincter and prevents reflux.

One laparoscopic technique that appears to show promise for GERD therapy involves approaching the esophageal sphincter from the outside, using laparoscopic surgical techniques, and performing a circumference reducing tightening of the sphincter by placement of an adjustable band such that it surrounds the sphincter. However, this procedure still requires surgery, which is more invasive than if an endogastric transluminal procedure were performed through the lumen of the esophagus or stomach. Furthermore, the necessity to provide for future adjustment in the band also requires some surgical access and this adjustment would be more easily made via a transluminal approach.

Further reading related to the pathophysiology of GERD includes “Mechanisms of Gastro-esophageal Reflux in Patients with Reflux Esophagitis,” New England Journal of Medicine 1982;307:1547-1552, Dodds W. J.; Dent J.; Hogan W. J.; Helm J. F.; Hauser R.; Patel G. K.; Egide M. S, “The Physiology and Patho-physiology of Gastric-emptying in Humans,” Gastroenterology 1984;86:1592-1610, and Minami H.; McCallum R. W., Gastro-esophageal Reflux—Pathogenesis, Diagnosis, and Therapy, Annals of Internal Medicine 1982;97:93-103, Richter J. E.; Castell D. O.

Evidence indicates that up to 36% of otherwise healthy Americans suffer from heartburn at least once a month, and that 7% experience heartburn as often as once a day. It has been estimated that approximately 1-2% of the adult population suffers from GERD, based on objective measures such as endoscopic or histological examinations. The incidence of GERD increases markedly after the age of 40, and it is not uncommon for patients experiencing symptoms to wait years before seeking medical treatment.

A need, therefore, remains for improved access technology, which allows a device to be transluminally introduced, advanced into the region of the mammalian gut, such as the esophagus or stomach, and implanted to perform tightening or adjustment of a portion of the mammalian gut, such as the esophageal sphincter. Ideally, the device would be able to be guided by fluoroscopy, ultrasound, MRI, CAT, or endoscopy. The device would further minimize the potential for injury to body lumen or cavity walls or surrounding structures. The device would further possess the capability for adjustment, both radially inward and radially outward using non-surgical, or external, methodology.

### SUMMARY OF THE INVENTION

Thus, it would be advantageous to develop systems and methods for placing an implant around a portion of a mammalian gut such that the implant may be implanted and adjusted within the body of a patient in a minimally invasive or non-invasive manner. An implant, a transluminal delivery system, and a method of use are provided according to embodiments of the inventions.

In one embodiment, the delivery system for placing an implant around a portion of a body lumen or cavity in the alimentary canal comprises an elongate tubular member having a sidewall, distal and proximal ends and at least one lumen extending therethrough and a piercing guide slidably axially positioned in said at least one lumen of said elongate tubular member. The piercing guide is capable of being extended radially outward from, or retracted radially inward into an aperture in a region near the distal end of the elongate tubular member and has a sharp distal end configured to penetrate tissue surrounding a body lumen. The piercing guide also includes a hollow lumen extending longitudinally therethrough. A pusher configured to axially move an elongate implant positioned in said piercing guide lumen relative to said piercing guide is slidably positioned in the hollow lumen of the piercing guide and axial movement is controlled by a control mechanism located at the proximal end of the delivery system. A coupler is located on the distal end of the pusher, said coupler being configured to releasably connect an implant to the pusher, wherein said release is controlled by a release mechanism located at the proximal end of the delivery system.

In one embodiment, a method of placing an implant around a portion of mammalian gut comprises inserting a delivery system, comprising an elongate tubular member having distal and proximal ends, a lumen extending therebetween, a distal and a proximal expandable member mounted near the distal end of the elongate tubular member and a hub connected to the proximal end of the expandable tubular member into a patient's esophagus, advancing the delivery system to a target treatment site in said patient's gut, such that the distal end of the elongate tubular member is adjacent the target treatment site and the distal expandable member is distal to the target treatment site and the proximal expandable member is proximal to the target treatment site, inflating the distal expandable member, inflating the proximal expandable member, drawing a vacuum in the region between the proximal and the distal expandable member to pull adjacent gut tissue toward the elongate tubular member, advancing a guide sleeve radially outward from an aperture in the elongate tubular member, said aperture located between the proximal and distal expandable member, puncturing the adjacent gut tissue with the distal tip of the guide sleeve, advancing the guide sleeve through the gut tissue so that the distal tip of the guide sleeve is located outside of the gut, and advancing an implant having a first, constrained linear configuration and a second, unconstrained circular configuration through the guide sleeve so that the implant is deposited around the external tissue or space adjacent to the gut, wherein the implant assumes said second circular configuration upon being advanced from said guide sleeve and dissects through the tissue external to the gut.

In an alternative embodiment, a method of placing of an implant within a portion of a mammalian gut, or alimentary canal, comprises inserting a delivery system, comprising an elongate tubular member having distal and proximal ends, a lumen extending therebetween, a distal and a proximal expandable member mounted near the distal end of the elongate tubular member and a hub connected to the proximal end of the expandable tubular member into a patient's esophagus, advancing the delivery system to a target treatment site in said patient's gut, such that the distal end of the elongate tubular member is adjacent the target treatment site and the distal expandable member is distal to the target treatment site and the proximal expandable member is proximal to the target treatment site, inflating the distal expandable member, inflating the proximal expandable member, drawing a vacuum in the region between the proximal and the distal expandable member to pull the gut tissue toward the elongate tubular member, advancing a guide sleeve radially outward from an aperture in the elongate tubular member, said aperture located between the proximal and distal expandable member, puncturing the gut tissue with the distal tip of the guide sleeve, advancing the guide sleeve partially through the gut tissue so that the distal tip of the guide sleeve is located between a first layer and a second layer of gut tissue, advancing an implant having a first, constrained linear configuration and a second, unconstrained circular configuration through the guide sleeve so that the implant is deposited in between the first and second layer of gut tissue, wherein the implant assumes said second circular configuration upon being advanced from said guide sleeve and in between said first and second layers of gut tissue.

n certain embodiments, the delivery system may be inserted through the pharynx of the patient and routed, antegrade, through the esophagus to the region of the entrance to the stomach. The delivery system may further include an endoscope to provide for endoscopic visualization of the body lumen or vessel through which the delivery system passes and to make further provision for visibility under fluoroscopic or ultrasonic monitoring. For example, the delivery system may permit visualization or measurement of the amount of residual opening in the lower esophageal sphincter (LES

In an alternative embodiment, an implant for adjusting a diameter of a portion of a mammalian gut comprises an outer sheath having a proximal end and a distal end, wherein the outer sheath is configured to assume a first, elongate shape when constrained and to transform to a second, substantially circular shape when unconstrained, a blunt dissecting tip located on the distal end of the outer sheath, a coupler located at the proximal end of the outer sheath, wherein the coupler is configured to releasably connect to a delivery system pusher, and an inner core comprising a shape memory material configured to adjust a diameter of the implant when the implant is in said second, unconstrained configuration and said shape memory material is activated.

In certain embodiments, the implant may have an inwardly curved bias, once released from the hollow piercing guide, to track along the circumference of the esophagus. The tip of the implant may be blunted, or bulbous, and capable of blunt dissection through tissue. The implant further is configured as having a curvature of at least 180 degrees of a circle so that it continues to follow the circumference of the outer wall of the esophagus as it is advanced. In certain embodiments, the implant may have a full 360-degree circular configuration. Alternatively, the implant may have a circumferential configuration that is greater than 360-degrees and allows for side-to-side overlap of adjacent members. In yet another embodiment, the implant can describe a coil with multiple turns and overlaps that are spaced to provide a substantially wider implant than would be obtained with a single 360-degree turn.

For purposes of summarizing the invention, certain aspects, advantages and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein. These and other objects and advantages of the present invention will be more apparent from the following description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1 is a front view schematic representation of the human upper digestive system including the esophagus and the stomach;

FIG. 2 is a front view schematic representation of the human upper digestive system with acid reflux occurring through an incompetent lower esophageal sphincter;

FIG. 3 is a front view schematic representation of the human upper digestive system with a delivery system advanced into the esophagus past the level of the lower esophageal sphincter, according to an embodiment of the invention;

FIG. 4 is a front view illustration of the lower esophagus and upper stomach with a delivery system placed therein and isolation balloons inflated, according to an embodiment of the invention;

FIG. 5 is a front view illustration of the lower esophagus and upper stomach with a hollow needle advanced radially from a trans-esophageal delivery system to penetrate the esophagus through to outlying tissue, according to an embodiment of the invention;

FIG. 6 is a front view illustration of the lower esophagus and upper stomach with an implant being advanced out of the hollow needle, according to an embodiment of the invention;

FIG. 7A is an illustration of the lower esophagus and surrounding tissue shown in lateral cross-section with an implant advanced circumferentially nearly completely thereabout, according to an embodiment of the invention;

FIG. 7B is an illustration of a portion of the mammalian gut shown in lateral cross-section with an implant disposed between layers of the portion of mammalian gut

FIG. 8 is an illustration of the lower esophagus and surrounding tissue shown in lateral cross-section with the delivery system removed and the implant remaining, according to an embodiment of the invention;

FIG. 9 is a frontal illustration of the upper gastrointestinal tract with a heating balloon inserted within an implant, according to an embodiment of the invention;

FIG. 10 is a side cross-sectional view of a delivery system distal end, according to an embodiment of the invention;

FIG. 11 is a side cross-sectional view of a delivery system proximal end, according to an embodiment of the invention;

FIG. 12 illustrates a longitudinal cross-sectional view of the distal region of a delivery system further comprising a pusher, an implant, and a coupler, according to an embodiment of the invention;

FIG. 13 illustrates an adjustable implant comprising a blunt dissecting distal tip, according to an embodiment of the invention;

FIG. 14 illustrates a top view of an adjustable implant comprising an internal steering mechanism, according to an embodiment of the invention;

FIG. 15 illustrates a lateral cross-sectional view of an implant comprising a shape-memory central support and a surrounding polymeric layer, according to an embodiment of the invention;

FIG. 16 illustrates a side view of the distal end of a delivery system comprising a guiding groove, according to an embodiment of the invention; and

FIG. 17 illustrates a frontal, cross-sectional, view of a stomach, with an implant placed around the region of the pyloric sphincter, according to an embodiment of the invention.

### DETAILED DESCRIPTION OF THE PREFERRED EMBODIMENTS

The present invention involves systems and methods for accessing a body lumen or cavity along the alimentary canal and controlling a diameter of the body lumen or catheter. In certain embodiments, a catheter or delivery system, may include an axially elongate hollow tubular member having a proximal end and a distal end. The axially elongate member further has a longitudinal axis and has one or more internal lumens that extend from the proximal end to the distal end for the passage of instruments, fluids, tissue, or other materials as well as delivery of an implant to the treatment site. The axially elongate hollow tubular member is generally flexible and capable of bending, to a greater or lesser degree, through one or more arcs in one or more directions perpendicular to the main longitudinal axis. As is commonly used in the art of medical devices, the proximal end of the device is that end that is closest to the user, typically a surgeon, or gastroenterologist. The distal end of the device is that end closest to the patient or that is first inserted into the patient. A direction being described as being proximal to a certain landmark will be closer to the user, along the longitudinal axis, and further from the patient than the specified landmark. The diameter of a catheter is often measured in “French Size” which can be defined as 3 times the diameter in millimeters (mm). For example, a 15 French catheter is 5 mm in diameter. The French size is designed to approximate the circumference of the catheter in mm and is often useful for catheters that have non-circular cross-sectional configurations. While the original measurement of “French” used π (3.14159 . . . ) as the conversion factor between diameters in millimeters (mm) and French, the system has evolved today to where the conversion factor is 3.0.

In certain embodiments, as will be described herein, the delivery system may be used to deliver an implant around the esophagus for tightening or adjusting the esophageal sphincter, for example to control GERD. Alternatively, the delivery system may be used to place an implant in between layers or around a portion of the stomach cavity for controlling the diameter of the portion of the stomach in an effort to reduce obesity. However, it is further envisioned that the methods and devices described herein may be used to access and treat the any body lumen or cavity along the mammalian alimentary canal, or gut, including the pyloric, duodenal, or other gastrointestinal sphincters, stomach cavity, or any other hollow organs or ducts. For example, the system and methods can be adapted for control of the pyloric sphincter at the distal end of the stomach cavity. The delivery system may be configured to deliver the implant through a wall of the body lumen and place the implant around and outer circumference of the body lumen, or in the tissue external to the body lumen. Alternatively, the delivery system may deliver the implant in between tissue layers of the body lumen.

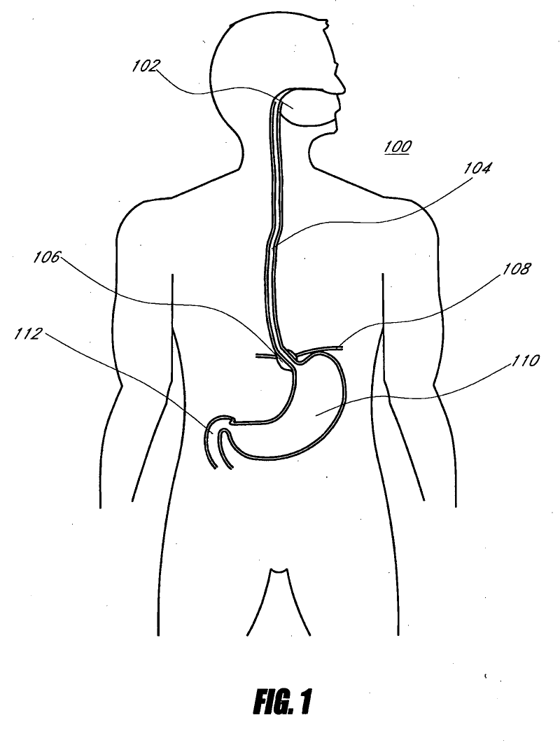

FIG. 1 is a schematic frontal (anterior) illustration (looking posteriorly) of a human patient 100 comprising an oral cavity, a pharynx 102, an esophagus 104, a lower esophageal sphincter 106, a diaphragm 108, a stomach 110, and a descending duodenum 112. In this illustration, the left anatomical side of the body of the patient 100 is toward the right of the illustration. FIG. 1 primarily illustrates components of the upper gastrointestinal, or digestive, tract.

Referring to FIG. 1, food enters the digestive system at the mouth (not shown) and enters the pharynx 102. It then travels, by swallowing and then peristaltic motion down the esophagus 104, through the lower esophageal sphincter 104 and into the stomach 110. After leaving the stomach 110, food passes through the descending duodenum 112 on its way to the small intestine (not shown) and large intestine (not shown). The lower esophageal sphincter 106 resides just at the level of the diaphragm 108, which is the muscular wall that separates the abdominal cavity from the thoracic cavity.

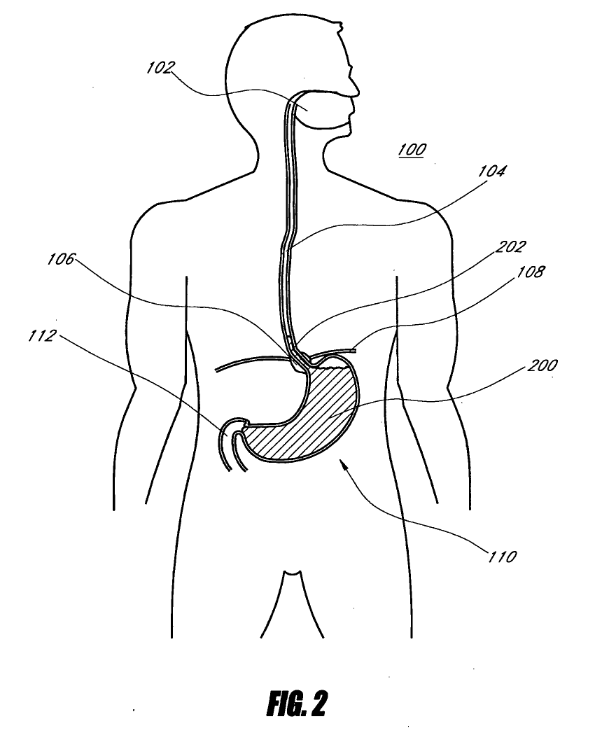

FIG. 2 is a schematic frontal illustration, looking posteriorly from the anterior side, of the patient 100 suffering from an incompetent lower esophageal sphincter 106. The gastrointestinal tract is shown with the pharynx 102, the esophagus 104, the lower esophageal sphincter 106, the diaphragm 108, the stomach 110 and the descending duodenum 112. Acidic stomach contents 200 are further shown. Regurgitated acidic material 202 or reflux of the stomach contents 200 are illustrated as residing in the lower part of the esophagus 104. While the stomach 110 is biochemically capable of handling the acidic fluids 200, the walls of the esophagus 104 are not so protected and will become damaged from repeated, or long-term, exposure to this reflux material 202.

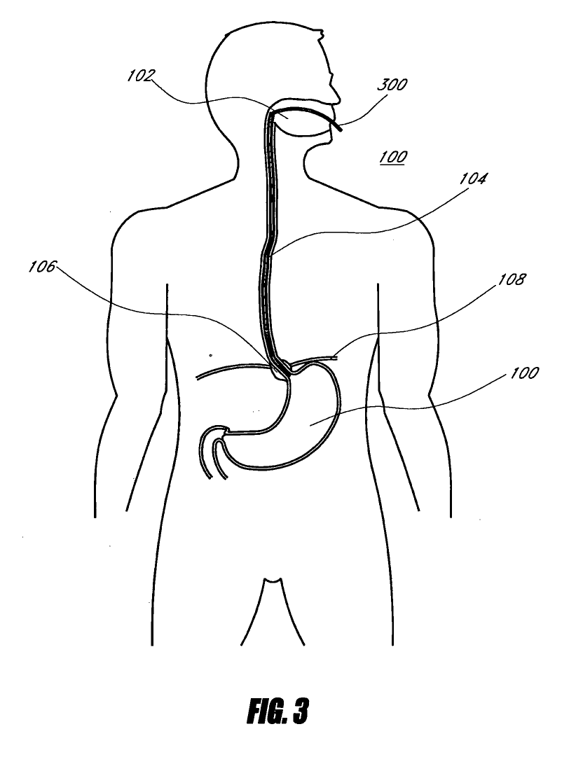

FIG. 3 is a frontal illustration of the patient 100 wherein a gastrointestinal transluminal catheter or delivery system 300 has been inserted into the esophagus 104 by way of the pharynx 102. The delivery system 300 has been inserted just into the stomach 110, having passed through the lower esophageal sphincter 106. The diaphragm 108 is also shown. The delivery system 300 may also be termed an endogastric catheter, trans-oral, or a trans-esophageally placed catheter. The proximal end of the delivery system 300 extends out of the patient such that it can be controlled by the attending physician while the distal end of the delivery system 300 may be located just downstream of the lower esophageal sphincter 106.

In certain embodiments, the delivery system 300 comprises a flexible structure, such that the delivery system may bend through angles at the back of the pharynx 102 where it passes into the esophagus 104 as well as through several less severe curves within the esophagus 104. For example, the delivery system may be configured to bend, articulate, or flex, around anatomical bends and be advanced into the region of the stomach, small intestine, or esophagus so that the longitudinal axis of its distal end is parallel to the esophageal, stomach, or intestinal axis. Provision can optionally be made to actively orient or steer the delivery system through the appropriate angles of between 0 to 90 degrees or more and to bend in one or even two planes of motion. The steering mechanism, in various embodiments, can be a plurality of pull-wires or pushrods, slidably disposed within internal lumens of the delivery system, or electromechanical actuators disposed on the exterior of the delivery system and electrically connected to control mechanisms at the proximal end of the delivery system, and the like. In most embodiments, the use of the delivery system eliminates the need for multiple access system components and allows completion of the procedure with a single instrumentation.

As will be further discussed below the steering mechanism is actuated, by the operator, by controls located at the proximal end of the sheath. The controls at the proximal end of the sheath are operably connected to the steering mechanism at the distal end of the sheath by linkages, pressure lumens, electrical lines, or the like, embedded within the sheath and routed from the proximal end to the distal end. In an embodiment, the structure of the delivery system is such that it is able to maintain a selectively rigid operating structure sufficient to provide stability against the esophagus and stomach to support the advancement of therapeutic instrumentation. For example, the elongate tubular member can be selectively stiffened, at least at its distal end, to provide a non-deflecting platform for support of instrumentation, which is passed therethrough

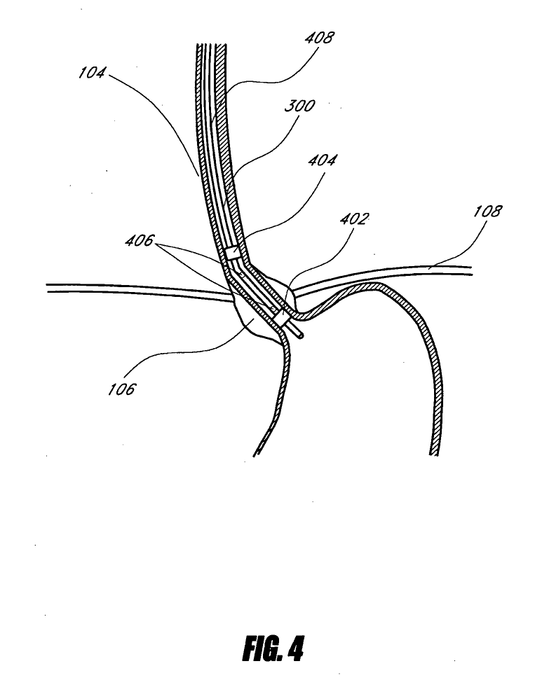

In certain embodiments, the delivery system 300 may further comprise one or more fixation devices for stabilizing the delivery system and maintaining the longitudinal position of the delivery system within the esophagus. The fixation device may be a selectively enlargeable structure that is expanded on the exterior of the delivery system portion that is resident within the esophagus. For example, the reversible fixation device may be an inflatable structure such as a balloon, a moly-bolt expandable structure, an expandable mesh, an umbrella, or the like, preferably positioned to expand within the stomach. In an embodiment, the fixation device is a balloon expanded on the exterior of the delivery system. The balloon inflation may be accomplished by injecting fluid into a port at the proximal end of the delivery system, the fluid pressure being transmitted through a lumen of the delivery system that operably connects the injection port to the interior of the balloon. At the completion of the procedure the balloon may be deflated and the delivery system be removed from the patient.

[For example, with reference to FIG. 4 the delivery system 300 may include a distal expandable member, or occlusion balloon, 402 and a proximal expandable member, or occlusion balloon, 404 attached to the distal region of the elongate tubular member 408. The occlusion balloons 402 and 404 are affixed, at least at each end, to the outer surface of the elongate tubular member 408 by bonds, which are created by a heat weld, a press-fit, an elastomeric seal, and the like. The balloon can be elastomeric and fabricated from materials such as silicone, polyurethane, latex rubber, C-Flex, and the like. The balloon can also be a non-compliant balloon and be fabricated from materials such as, but not limited to polyester, nylon, polyethylene, irradiated polyethylene, and the like.

In use, the proximal and distal occlusion balloons 402 and 404 seal the annulus between the elongate tubular member and the body lumen wall against the passage of fluids such as air, stomach acid, water, and the like. The occlusion balloons have an internal volume that may be inflated or deflated through apertures (not shown) in the wall of the elongate tubular member 408 of the delivery system 300. The apertures are operably connected to one or more inflation lumens (not shown) within the delivery system 300, such that the inflation lumen(s) may provide fluid communication between a connection port on the proximal end of the delivery system 300 to the apertures. The inflation lumens may carry injected saline, air, radiographic contrast media, water, or the like, under pressure to inflate or deflate the occlusion balloons 402 and 404.

In certain embodiments, the delivery system 300 may further comprise one or more vacuum ports 406 and disposed intermediate the proximal occlusion balloon 404 and the distal occlusion balloon 402. The vacuum port(s) 406 have an opening on the outer surface of the delivery system 300 and are operably connected to vacuum lumens (not shown) within the delivery system 300. In use, the vacuum lumens may transport fluid into or out of the body lumen via the vacuum ports 406. The vacuum lumens are operably connected to vacuum access ports on the proximal end of the delivery system 300, such as a luer lock, luer, bayonet, threaded, swage lock, pushbutton quick-connect, or any other suitable type of connection known in the arts.

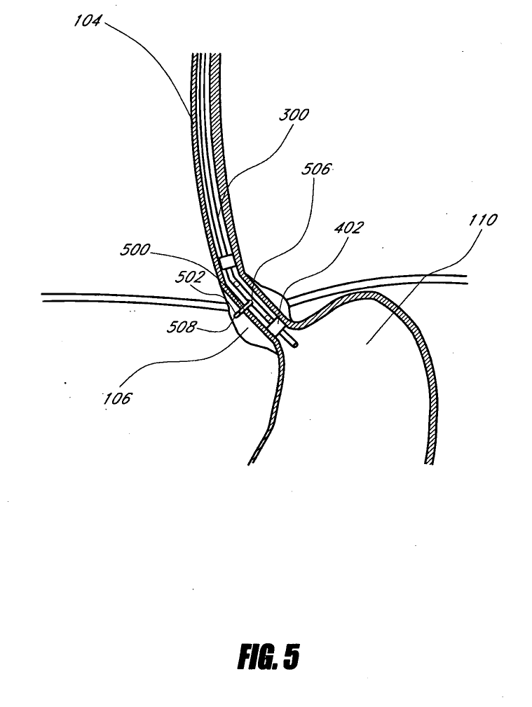

The delivery system 300 further comprises a piercing guide, slidably insertable within a lumen of the delivery system 300, for puncturing the wall of a body lumen adjacent to the delivery system 300. For example, as shown in FIG. 5, a guide sleeve or hollow piercing guide (or “needle guide”) 500 may be advanced radially from the delivery system 300 to penetrate the esophagus 104 through to outlying tissue 106, in this case the lower esophageal sphincter 106. In certain embodiments, the needle guide may include further a deflection mechanism at its distal end such that the needle guide can be circumferentially aligned with the exterior of the esophagus wall.

Here, the elongate tubular member 408 further comprises a needle lumen (not shown) extending from the proximal end of the elongate tubular member 408 to an aperture, or needle guide port, 506 located in a sidewall at the distal region of the elongate tubular member 408. The needle guide 500 is slidably positioned within the needle lumen such that it may be advanced through the needle lumen and exit the delivery system 300 via the needle guide port 506. The hollow needle guide 500 further comprises a needle pusher (not shown) within the needle lumen. The needle pusher is permanently affixed, at its distal end, to the hollow needle guide 500 and at its proximal end to a needle advance lever, handle, knob, motor, jackscrew, or other advancing mechanism. When the needle pusher is retracted proximally, the hollow needle guide 500 is retracted and is pulled entirely within the tubing of the delivery system 300. Conversely, when the needle pusher is advanced, the needle guide 500 is advanced from the distal end of the delivery system 300 through the needle guide port 506.

The guide sleeve or hollow needle guide 500 comprises a central lumen 502 and a sharp, distal tip 508. The sharp point on the distal tip 508 may be created by beveling the distal tip of the hollow needle guide 500. The bevel is between 20 and 70 degrees with respect to the longitudinal axis of the hollow needle 500. In certain embodiments, the needle guide 500 comprises a distal tip 508 that is non-coring. The needle guide 500 may be constructed of polymers such as glass-filled polycarbonate, or, preferably, from nitinol or other shape memory alloy.

In certain embodiments, for example, wherein the needle guide 500 is comprised of a shape memory material, the distal tip 508 may be manipulated using Ohmic heating of the needle guide 500. Shape memory materials exist in two distinct solid phases called martensite and austenite. The martensite phase is relatively soft and easily deformed, whereas the austenite phase is relatively stronger and less easily deformed. In certain embodiments, the shape memory needle guide 500 may processed to form a memorized shape in the austenite phase in the form of approximately a 90° arc. The shape memory alloy is then cooled to enter the martensite phase and deformed into a substantially linear shape to be advanced through the delivery system 300. Thus, when the needle guide 500 is heated above its austenite finish temperature, the heating causing the needle guide 500 to assume increasingly austenitic conditions and transform to the pre-set austenite shape, for example approximately a 90° arc. In one embodiment, the austenite finish temperature is approximately 30° C., alternatively the austenite finish temperature may be in a range between 22° C. and 50° C., alternatively between 30° C. and 45° C.

Alternatively, the needle guide may comprise a super-elastic shape memory alloy having a pre-formed configuration such as 90° arc. In use, the super elastic needle guide may be deformed into a substantially linear configuration by the pressure exerted from walls of the lumen of the elongate tubular member. However, once advanced from the needle guide port in the elongate tubular member, the needle guide will resume its pre-formed shape of an arc.

In an alternative embodiment, the hollow needle guide 500 may comprise a deflecting tip, which is articulated, automatically or by the user through controls at the proximal end, to curve so that the outlet is approximately 90 degrees from the longitudinal axis of the hollow needle guide 500 where it exits needle port 506. The articulation can be performed by use of pull wires or pushrods slidably disposed within the hollow needle guide 500. In another embodiment, the needle guide 500 may be pre-curved and advanced outwardly in arc-like fashion. Here, the needle guide 500 is not moved outward at 90 degrees to the axis of the delivery system, but rather in an arc that spirals radially outward as it translates circumferentially around the delivery system.

In use, the needle guide 500 is advanced radially outward from the needle guide port 506 and the sharp, distal tip 508 penetrates through the esophageal wall into the region exterior thereto, and is deflected so that the opening at the distal end of the needle guide 500 is aligned tangentially with the circumference of the esophagus. In certain embodiments, the needle guide 500 is not moved outward at 90 degrees to the axis of the delivery system, but rather in an arc that spirals radially outward as it translates circumferentially around the delivery system. In certain embodiments, the needle guide 500 may not completely break through the wall of the body lumen, such as the esophagus, to the exterior, but instead only penetrate partially through the body lumen wall such that an implant may be delivered between the tissue layers of the body lumen wall

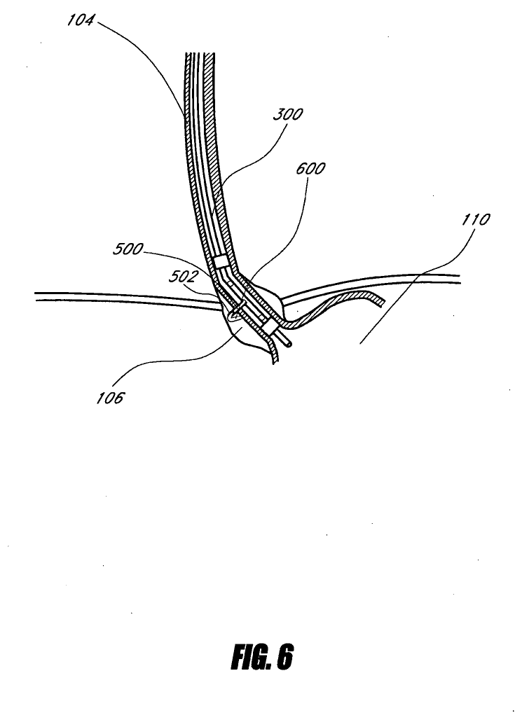

Once the needle guide 500 has penetrated the esophageal wall, an implant may be delivered via the needle guide lumen 502. As shown in FIG. 6, implant 600 may be advanced out of the hollow needle guide 500 of the delivery system 300. In one embodiment, the distal tip of the implant 600 is rounded, with no sharp edges, so as to permit blunt dissection of the tissue 106 as the implant 600 is pushed out of the needle guide 500.

In certain embodiments, the implant 600 may have a pre-determined shape implant. For example, while in the delivery system 300 or the hollow needle guide 500, the implant 600 may be compressed and forced to take the shape of the lumen within which it resides. However, when the implant 600 is advanced out of the hollow needle guide 500, it may take on its pre-determined shape, for example, a split ring, a “C” shape, or other configuration with a pre-specified neutral diameter.

For example, in one embodiment, the implant 600 may comprise in part nitinol or any other shape memory material. The implant 600 can be fabricated from shape memory materials such as nickel-titanium alloy (nitinol). The implant 600 may further be a composite structure of nitinol, stainless steel, polymers, including shape memory polymers, bioresorbable polymers, and the like. In certain embodiments, the implant may be configured as a band with its width being wider than its thickness. The edges of the band can comprise elastomeric or polymeric materials that serve as a strain relief and minimize tissue erosion in the presence of the implant. The implant may also include radiopaque markers, which denote its ends and at least some positions on its intermediate length. The implant is generally stiff so that circumferentially applied forces do not cause the implant to bend, buckle, or become distorted during placement or advancement. In certain embodiments, the implant can be constructed as a composite structure with an external sleeve and a replaceable core. For example, the external sleeve can be constructed of stainless steel with a malleable, fully annealed structure. A core rod can be inserted into the central lumen of the external sleeve. The core can be fabricated from nitinol and, when heated, bias the sleeve to constrict diametrically or expand diametrically, depending on the heat treatment and fixturing parameters. A contracting core rod can be removed and be replaced with an expanding core rod, if the patient care so requires.

The proximal end of the implant 600 is releasably affixed to the distal end of a pusher by a releasable coupler (not shown). The pusher is configured to translate axially with substantial force and convey and move the implant under said substantial force. The pusher is controlled at the proximal end of the delivery system. In use, the pusher forcibly advances the implant 600 out of the delivery system 300 and forces it along its blunt dissecting path through the tissue surrounding the esophagus. In another embodiment, the pusher is a rotational device that is powered by manual or assisted rotation of a knob at the proximal end of the delivery system, or by an electromechanical actuator within the delivery system. The assisted rotation can be an actuator such as an electromechanical motor, pneumatic cylinder, hydraulic cylinder, or the like. Rotation of the pusher spools the implant, which is wrapped around a hub or reel, out of the hollow needle.

The releasable coupler is operably connected to a release mechanism located at the proximal end of the delivery system 300 such that the release of the implant may be controlled by a deliberate action at the proximal end of the delivery system, said action being transmitted along the length of the delivery system 300 by a mechanical, electrical, hydraulic, pneumatic, magnetic or any other suitable type of linkage.

The implant 600 may have a lateral cross-sectional shape that is round, elliptical, rectangular, triangular, oval, “H” shaped, “U” shaped, flat, flat with reinforcing longitudinal ridges, or the like. The implant may further be comprised of a shape memory material. For example, in one embodiment, the implant 600 may comprise a plurality of nitinol core members, each with different memory shapes. In another embodiment, the implant 600 may comprise a shape-memory outer sleeve and standard elastomeric or malleable core structures fabricated from materials such as, but not limited to, stainless steel, tantalum, platinum, gold, iridium, titanium, and the like. The implant 600 may further comprise an outer coating of polymeric origin. Materials suitable for coating the implant include, but are not limited to, polytetrafluoroethylene, polyester, polyamide, polyurethane, hydrogel, thermoplastic elastomer, fluorinated ethylene propylene, and the like. The polymeric materials can further be impregnated with drugs or chemicals that promote healing, resist or promote thrombosis, resist infection, promote volume swelling, or promote lubricity. In another embodiment, the implant 600 has a gas port that exits at or near the distal tip of the implant 600. Carbon dioxide gas, or other suitable gas, can be injected into the implant 600 through the delivery system 300 such that it exits at the distal tip of the implant 600 and assists with blunt dissection of the tissue as the implant 600 is deployed.

The implant may be further be configured as an arc comprising at least 180° of a circle so that it continues to follow the circumference of the outer wall of the esophagus as it is advanced. In some embodiments, the implant may have a full 360° circular configuration. Alternatively, the implant may have a circular configuration that is greater than 360° and allows for side-to-side overlap of adjacent coils. Alternatively, the implant may have comprise multiple coils wherein the adjacent coils are spaced apart to provide a substantially wider implant than would be obtained from a single 360° circular implant.

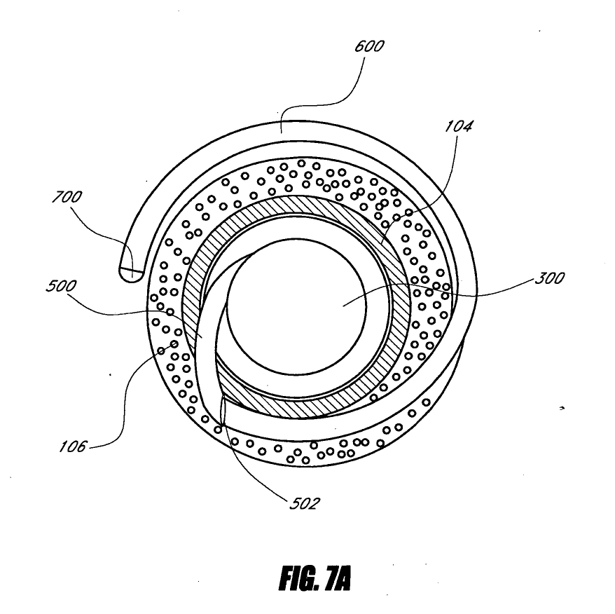

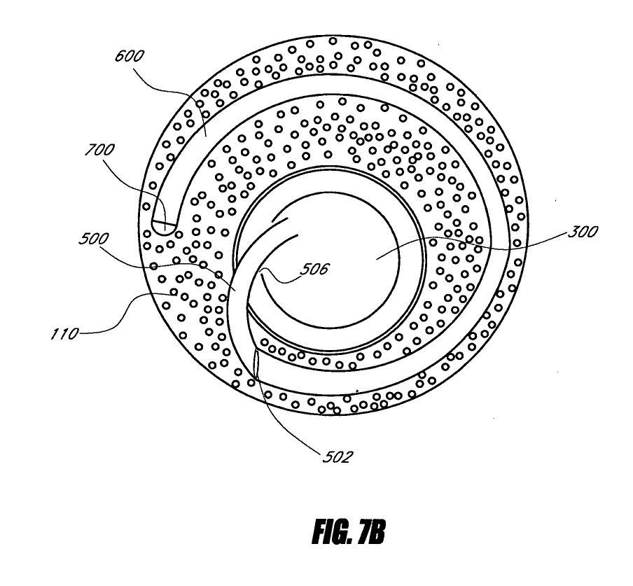

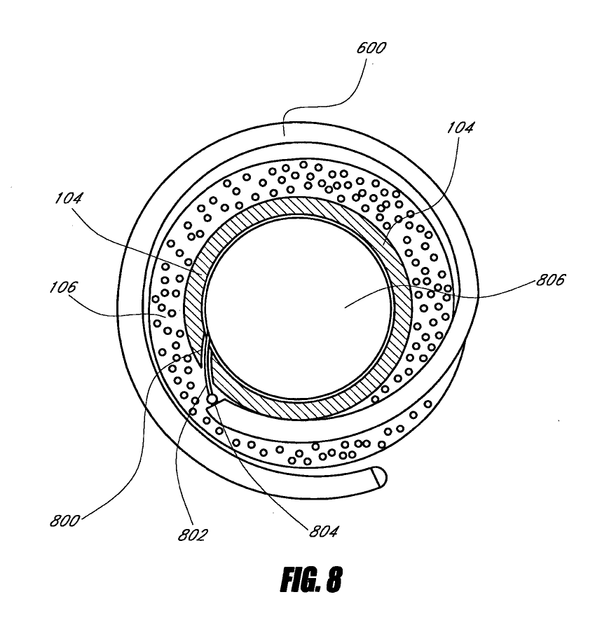

FIG. 7A is an illustration of the lower esophagus 104 and surrounding tissue 106 shown in lateral cross-section with the implant 600 being advanced through the surrounding tissue 106. Here, the needle guide 500 has punctured through the wall of the esophagus to creating an opening in the esophageal wall. The implant 600 is then advanced through the lumen 502 of the needle guide 500. The implant 600 is expelled into the lower esophageal sphincter 106 and is forcibly advanced through the tissue 106 by blunt dissection. The distal tip 702 of the implant 600 is rounded or tapered and is not sharp, such that the distal tip 702 is incapable of cutting through tissue such as blood vessels, skin, and the like. However, under longitudinal pressure, the distal tip 702 is capable of bluntly dissecting through layers of muscle such as that comprising the lower esophageal sphincter 106. The inner wall 702 of the esophagus 104 is also illustrated, said inner wall 702 comprising mucosa and submucosa. Here, the implant is advanced completely through the esophageal wall such that once delivered, the implant will be at least partially surround an outer circumference of the esophagus and will reside between the exterior wall of the esophagus and the visceral peritoneum, or lining of the abdominal cavity as shown in FIG. 8.

With reference to FIG. 8, the needle guide 500 (not shown) has been retracted into the delivery system 300. The puncture wound 800 remains to heal on its own accord or to be closed from the inside by way of standard closure devices such as polymeric plugs, sutures, or the like. The delivery system 300 is not shown since it has been withdrawn from the lumen 806 of the esophagus 104. The implant 600 further comprises a coupler 802 affixed to the proximal end of said implant 600. Here, the implant 600 circumnavigates in excess of 360 degrees of the esophagus 104 but less than 720 degrees. In alternative embodiments, the implant 600 may circumnavigate about 180 degrees or more of the esophagus 104, i.e. at least one half turn, or alternatively up to 10 turns around the esophagus. The coupler 802 is either integral to or separately attached to the proximal end of the implant 600 by welding, friction fit, interference fit, adhesive bonding, or the like. The coupler 802 is configured with a grasping detent or undercut 804 that permits the delivery system pusher (not shown) to releasably grasp the coupler 802. The implant 600 and the coupler 802 comprise similar materials on their outer surfaces to minimize any electrochemical effects or corrosion. Furthermore, the implant 600 and the coupler 802 further comprise at least one radiopaque marker (not shown). The radiopaque marker comprises materials such as, but not limited to, platinum, gold, tantalum, iridium, barium sulfate, bismuth salt, or other radio-dense material. The radiopaque marker can be affixed to the exterior of the implant 600 or it can be affixed internally so that it is not exposed on the exterior of the implant 600. Preferably the proximal end and the distal end of the implant 600 comprise a radiopaque marker and in another embodiment, substantially the entire length implant 600 is radiodense. The implant 600 can also be made to be visible under ultrasound and it is further capable of magnetic resonance imaging (MRI) without heating or moving since it comprises non-magnetic materials.

In certain embodiments, the delivery system may comprise a tissue closure apparatus to actively close the hole, or approximate the tissue, in the esophageal wall following retraction of the hollow needle guide. Such tissue closure apparatus includes lasso devices, sutures, staples, fibrin plugs, polymeric plugs fabricated as rigid, foam, gel, or the like. When the hollow needle is retracted within the delivery system, the tissue closure apparatus is actuated to close the fenestration, should that be necessary. Examples of tissue closure apparatus include those cited in U.S. Pat. Nos. 6,527,734 to Cragg et al., 5,746,755 to Wood et al, 5,417,699 to Klein et al, 5,700,273 to Buelna et al, 5,445,597 to Clark et al, and 6,425,901 to Zhu et al, the entirety of which are hereby included herein by reference.

Alternatively, as shown in FIG. 7B, the delivery system may be configured to deliver an implant in between layers of the tissue of a body lumen, such as the stomach or any other lumen or cavity along the mammalian gut. Here, the needle guide 500 is advanced to penetrate the wall of the stomach cavity 110. The needle guide is not advanced entirely through the wall of the stomach cavity 110, however, but positioned in between the tissue layers of the stomach wall. The implant 600 may then be advanced through the needle guide lumen. 502 into between the layers of stomach tissue 110. As discussed above, the blunt tip 702 of the implant is capable of bluntly dissecting through the layers of tissue or muscle in the stomach wall. The implant 600 is curved such that as the implant is longitudinally advanced from the needle guide lumen 502, it carves a path through between adjacent tissue layers and becomes implanted within the wall of the stomach cavity. Once fully deployed, the implant 600 surrounds a circumference of the stomach cavity and is sandwiched between layers of the stomach tissue 110. In certain embodiments, the delivery system may further include a tool for grasping the wall of the stomach cavity as the implant is threaded through to provide tension and thereby prevent perforation of the stomach or the implant from piecing entirely through the stomach. Once the implant has been fully deployed within the wall of the stomach cavity, the needle guide 500 may be retracted into the delivery system.

In certain embodiments, the delivery system may further include apparatus to monitor the progress of the implant delivery. For example, the progress of the delivery can be monitored by affixing a small permanent magnet at the distal tip of the implant. An array of Hall-effect sensors may be distributed about the circumference the head of the delivery system so that the position of the magnet can be detected by the circumferential array of sensors. The position information regarding the distal tip of the implant can be transmitted through electrical lines to processing and display apparatus at the proximal end of the delivery system. Alternatively, a simple linear scale may be provided on the pusher so that the amount of pusher projection is visualized at the proximal end of the delivery system by a scale affixed to apparatus affixed to the proximal end of the pusher linkage, which operably connects the pusher to forcing apparatus at the proximal end of the delivery system.

In certain embodiments, a diameter of the implant 600 can be adjusted after implantation. For example, if the implant 600 comprises a shape memory material, as discussed above, the adjustment can be accomplished by Ohmic, or resistive, heating of the implant for example by heating with a hot balloon, by bombarding the implant with high intensity focused ultrasound (HIFU), by radio frequency (RF) bombardment, by microwave bombardment, or any other suitable energy. Alternatively, the implant may be adjusted in a direction opposite that caused by heating by cooling the implant to transform the implant to its malleable martinsite phase and then imparting mechanical force to provide such opposite coercion.

For example, in one embodiment, the implant may be fabricated from shape memory nitinol with an austenite finish temperature of 42° C. Following implantation, 2 weeks is a reasonable minimum delay time to allow for healing, a balloon catheter may be inserted trans-esophageally into the patient's alimentary canal and advanced so that the balloon resides inside the implant. The balloon may then be inflated with hot water to heat the implant causing the implant to become increasingly austenitic and causing the implant to constrict diametrically. The diameter of the heating balloon is the same as the desired diameter of the implant. Furthermore, the balloon pressures can be kept low so as not to prevent radial constriction of the implant. The longer the heat is applied, the further constriction occurs. Alternatively, a balloon that is expandable to a larger diameter may be used to allow for re-expansion of the implant. Here, the balloon is filled with cold water to cool the implant and cause the implant to become martensitic. The implant may require cooling to temperatures below those initially required for maintenance of martensitic conditions due to hysteresis in the cooling curve. Once in the martensite phase, the implant becomes soft and malleable and can be adjusted outward by expansion of the balloon.

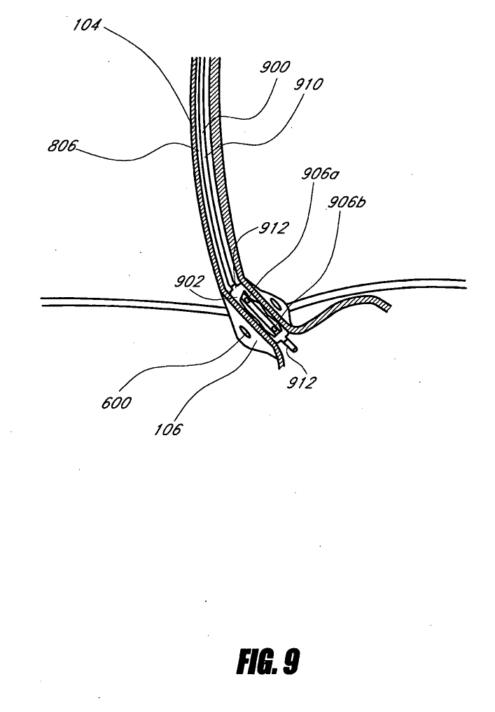

FIG. 9 illustrates the distal end of a balloon catheter 900, which as described above, may be used in certain embodiments to heat or cool the a shape memory implant 600 after it has been delivered to the treatment site in order to adjust the size and/or shape of the implant. The balloon catheter 900 is inserted into the lumen 806 of the esophagus 104. The balloon catheter 900 includes a catheter shaft 910 and a balloon 902 fabricated from materials such as, but not limited to, polyurethane, silicone elastomer, thermoplastic elastomer, latex rubber, or the like. The balloon catheter can, in another embodiment, comprise a balloon 902 which is nondistensible and fabricated from materials such as, but not limited to, polyester, polyamide, polyimide, irradiated polyethylene, and the like. The balloon 902 has a thin wall and is capable of being inflated through lumens (not shown) within the balloon catheter 900 that are exposed to the interior of the balloon by apertures 906 communicating between the lumen and the interior of the balloon 902. The proximal end (not shown) of the catheter 900 comprises a plurality of inflation ports (not shown) suitable for inflating the balloon 902 with pressurized fluid such as, but not limited to, water, saline, radiopaque contrast media, refrigerant, or the like. The balloon 902 is generally axially symmetric and is bonded at each end to the catheter shaft 910 by a plurality of bonds 912. The plurality of inflation ports are suitable for infusion of pressurized fluid into the lumens of the catheter 900 and the balloon 902 such that a continuous flow of fluid is maintained to deliver the desired amount of heat or cooling to the balloon 902 so that the balloon 902 can operably transfer heat to or from the implant 600. An external heater and pump (not shown) is operably connected to the inflation ports to generate the flow of thermal pressurized fluid within the balloon 902. In certain embodiments, the catheter shaft 910 can be surrounded by a sheath (not shown), or other material to provide insulation for the esophagus 104, as heat is being added or withdrawn to the balloon 902. A first isolated lumen in catheter 900 is used for fluid input and that lumen is operably connected through aperture 906a into the interior of the balloon 902. A second isolated lumen is operably connected to a separate second aperture 906b and is used to drain fluid from the interior of the balloon 902.

Referring to FIG. 9, the balloon 902 is expanded within the esophagus 104 and delivers or withdraws heat from the implant 600 embedded around the esophagus. By withdrawing heat and expanding to a diameter larger than that of the lumen 806 of the esophagus 104, the balloon 902 lowers the temperature of the shape memory implant 600 below martensitic start temperature and makes the implant increasingly malleable. The balloon 902 further provides radially outwardly directed force to deform the implant 600 and expand the now somewhat malleable implant to a larger diameter. Lowering the temperature below martensite finish temperature maximizes the malleable properties of the implant 600, although consideration is made not to cool the adjacent tissue too much so as to cause irrecoverable damage. Conversely, pumping heated fluid through the balloon 902 heats the shape memory implant 600 and raises the temperature of the implant increasingly above its austenite finish temperature which may cause the implant to assumes its pre-set shape memory having a deceased diameter. In an alternative embodiment, the heating can also be generated externally using HIFU or internally using microwaves, radio frequency heating, or the like.

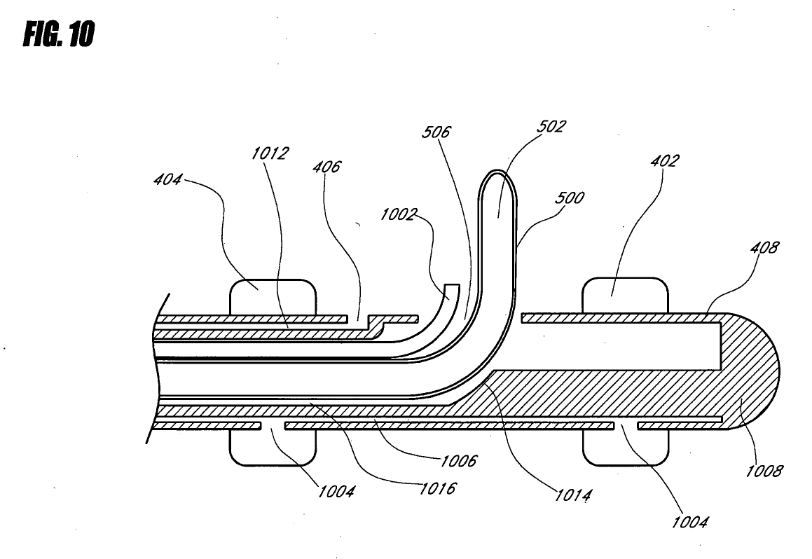

FIG. 10 illustrates the distal end of one embodiment of the delivery system 300 in longitudinal cross-sectional view. The distal end of the delivery system 300 includes an elongate tubular member 408, a distal occlusion balloon 402, a proximal occlusion balloon 404, a vacuum port 406, a vacuum lumen 1012, a plurality of balloon inflation apertures 1004, a balloon inflation lumen 1006, a catheter tip 1008, an endoscope 1002, a deflector 1014, a needle guide 500 further comprising a central guide lumen 502, and a needle guide port 506. The vacuum lumen 1012, the balloon inflation lumen 1006, and the instrument lumen 1016 are integrally formed with the delivery system tubing 408. The elongate tubular member 408 may be extruded, co-extruded, or laid up as a composite structure to form the basic tubing configuration. The balloon apertures 1004 and the vacuum port 406 are drilled, cut, melted, or otherwise formed in the wall of the tubular member 408 and operably communicate with the balloon inflation lumen 1006 and vacuum lumen 1012, respectively. The needle guide port 506 is similarly cut into the wall of the tubular member 408 to operably communicate with the instrument lumen 1016. The needle guide port 506 is generally located in the same axial region as the deflector 1014. In certain embodiments, the delivery system may further include a deflector 1014 to steer the needle guide 500 through the needle guide port 506. The deflector 1014 can be integrally formed with the tubular member 408 or it can be a separate structure. The deflector 1014 can further be movable or hinged and be operably connected to a manipulator at the proximal end of the delivery system 300 by a linkage, wire, pushrod, electrical connection, or the like. The distal tip of the elongate tubular member may include a nose cone 1008 which can be separately formed using injection molding, liquid injection molding, and the like, and be welded or adhered to the tubing 408 or it can be integrally formed using RF forming, ultrasonic forming, induction heating, and the like.

In an embodiment, the balloons 402 and 404 are elastomeric and fabricated from materials such as, but not limited to, polyurethane, silicone elastomer, thermoplastic elastomer, latex rubber, or the like. In another embodiment, the balloons 402 and 404 are non-compliant and are fabricated from materials such as those used to fabricate angioplasty balloons, including but not limited to, irradiated polyethylene, polyester, polyimide, polyamide, copolymers of the aforementioned, and the like. The non-compliant balloons are generally stretch blow-molded to achieve highly oriented polymeric structures and attendant high wall strengths. The balloons 402 and 404 are generally cylindrical and have cylindrical bond areas with lengths of between 1 and 50-mm. The balloons 402 and 404 have a wall thickness ranging from 0.0005 inches to 0.020 inches, depending on materials used for construction. The balloon diameters range between 0.5 inches and 2.0 inches while the lengths range between 0.5 inches and 3 inches.

The needle guide 500 comprises a sharpened or pointed end and is a hollow axially elongate structure having a central lumen 502. The needle guide 500 can be pre-shaped to curve in a specific direction or configuration when it is advanced out of a constraint such as the instrument lumen 1016 comprised within the delivery system 300. In the illustrated embodiment, the needle guide 500 is advanced axially to project out the side of the delivery system 300. In another embodiment, the needle guide 500 may be coiled or wound around the axis of the delivery system 300 such that rotation of a spindle or hub advances the needle guide 500 radially outward. The rotation can be generated by an electric motor, pneumatic force, hydraulic force, or by a torque shaft extending from the coiled needle guide 500 all the way to the proximal end of the delivery system 300 where it is terminated by a lever or knob which can be manually turned to generate the rotation.

In certain embodiments, the delivery system may further include an endoscope 1002. The endoscope 1002 can be a commercially available endoscope with side view capability or it can have a flexible distal end and comprise articulation capability such that its distal tip can be turned substantially perpendicular to the axis of the delivery system to view radially outward through the needle guide port 506. The endoscope may provide for visualization of the body lumen or vessel through which it passes and may further provide for visibility under fluoroscopic or ultrasonic monitoring. In addition, the endoscope may permit visualization of or measurement of residual opening in an adjacent sphincter to assess the amount of adjustment needed.

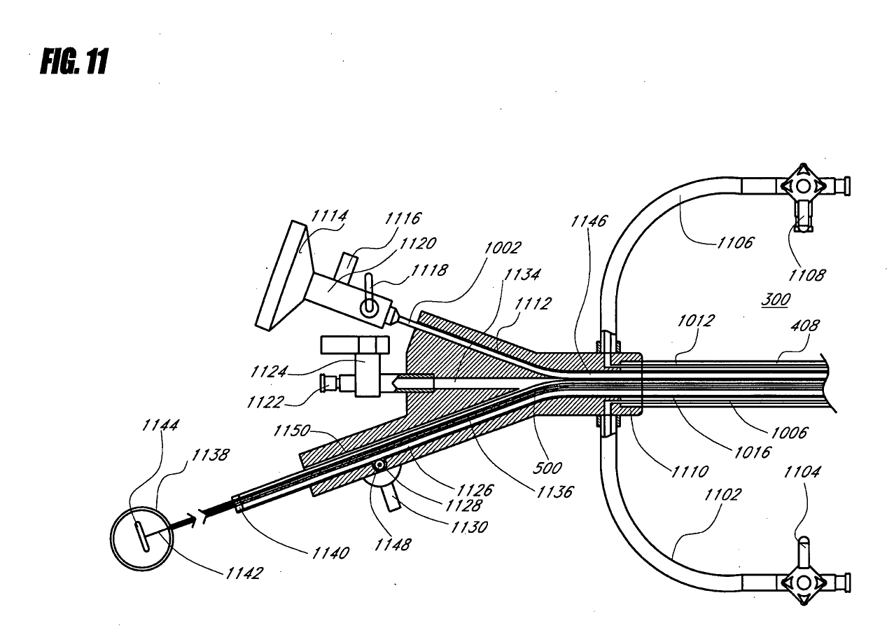

FIG. 11 illustrates the proximal end of an embodiment of the delivery system 300, shown in longitudinal cross-sectional view. The proximal end of the delivery system 300 comprises the tubular member 408, further comprising the vacuum lumen 1012, the balloon inflation lumen 1006, and the instrument lumen 1016. The delivery system 300 also comprises a delivery system hub 1110, the needle guide 500, an implant pusher 1136, an implant pusher handle 1138, a coupler release handle 1144, a coupler linkage 1142, a fluid seal 1140, a needle guide rack 1126, a needle guide pinion gear 1128, a needle guide advance lever 1130, a fluid infusion lumen 1134, a flushing port 1122, a flushing stopcock 1124, an endoscope 1002, an endoscope lumen 1112, an endoscope eyepiece 1114, an endoscope hub 1120, an endoscope articulating lever 1118, a flexible endoscope catheter 1146, and an endoscope light port 1116. The delivery system 300 further comprises a vacuum delivery line 1106, a vacuum valve 1108, a balloon inflation line 1102, and a balloon inflation valve 1104.

Referring to FIG. 11, in one embodiment, the elongate tubular member 408 may be extruded with the vacuum lumen 1012, the balloon inflation lumen 1006, and the instrument lumen 1016 integrally formed during the extrusion. Alternatively, the delivery system tubing 408 may also be composite tubing fabricated, for example, with an outer layer, a reinforcing braid or coil, and an inner layer, the inner layer being fused to the outer layer through holes or fenestrations in the reinforcement. The tubular member 408 can further comprise longitudinal fibers fabricated from materials such as, but not limited to, polyester, polyimide, Kevlar™, or the like, to impart stretch resistance, or bars to provide additional column strength. The tubular member 408 can further comprise an exoskeleton of flexible interlocking members (not shown) to provide kink resistance and high flexibility as well as column strength. The delivery system tubing 408 is affixed, at its proximal end, to a delivery system hub 1110, which is a molded or machined part. The delivery system hub 1110 is affixed to, and its lumens operably connected to, the tubular member 408 by solvent bonding, insert molding, adhesive bonding, welding, or similar process. The delivery system hub is affixed to, and operably connected to the lumens of the vacuum line 1106 and the balloon inflation line 1102. The balloon inflation line 1102 and the vacuum line 1106 are affixed to valves or stopcocks 1104 and 1108, respectively, with the lumens of each line 1102 and 1106 being operably connected to the through lumens of the valves or stopcocks 1104 and 1108. The length of the balloon inflation line 1102 and the vacuum line 1106 can range between 0 and 25-cm, with the lower limit describing an embodiment where the valves 1104 and 1108 are affixed directly to the hub 1110. The lengths of the balloon inflation line 1102 and the vacuum line 1106 can be the same, or they can be different. The instrument lumen 1016 can be a single lumen through which the endoscope catheter 1146 and the needle guide 500 are slidably constrained to axial or rotational motion, or it can be a multi-lumen channel, one lumen being adapted for each instrument passed therethrough. The instrument lumen 1016 divides within the hub 1110 to form an endoscope lumen 1112, a fluid infusion lumen 1134, and a needle guide lumen 1150, each of which operably continue to the proximal end of the hub. The proximal end of the fluid infusion lumen 1134 is affixed to, and operably connected to, the stopcock or valve 1124, which is terminated with the fluid infusion or flushing port 1122.

The hub 1110 comprises components to control the axial movement of the needle guide 500 such that a mechanical advantage is imparted on the axial travel of the needle guide 500. The needle guide control components include the rack gear 1126, which is affixed to the outer surface of the needle guide, the pinion gear 1128, which is affixed to the hub 1110 by an axle or rod 1148, thus permitting only rotational motion, and the control lever 1130, which is affixed to the pinion gear 1128. The control lever 1130 is manually moved by the operator, or is moved by an electric motor, hydraulics, pneumatics, or other powered device (not shown). The function of the rack and pinion gear can be replaced with a jackscrew, where a knob, longitudinally constrained from motion, but provided with freedom to rotate relative to the hub 1110, is rotated around the longitudinal axis of the needle guide 500 so as to move a threaded region on the needle guide. Referring to FIGS. 5 and 11, the needle guide 500 comprises a lumen 502 through which the pusher 1136 is disposed and constrained to axial or rotational movement. The pusher 1136 is, in an embodiment, fabricated from tubing with a central lumen to allow for passage of instruments therethrough. A seal 1140, at the proximal end of the needle guide 500, prevents fluid from entering the needle guide lumen 502 around the pusher 1136. The pusher 1136 is welded, adhesively bonded, clamped to, or otherwise affixed to the pusher handle 1138. The pusher 1136 is configured to translate with substantial force of between 0.5 and 200 pounds and preferably between 1 and 50 pounds. In an embodiment, the proximal end of the pusher is configured to be controllably moved by a jackscrew, handle and lever with ratchet, or other device with mechanical advantage (not shown)) to advance the pusher. The coupler linkage 1142 is slidably constrained to axially, or rotationally, move within the pusher 1136 and is affixed, at its proximal end, to a coupler release handle 1144. Another seal or valve (not shown) can be placed at the proximal end of the pusher tubing 1136 to prevent fluid passage around the coupler linkage 1142. Seals can also be placed at the proximal end of the needle guide lumen 1150 and the endoscope lumen 1112, to prevent the passage of fluids into or out of the hub 1110 around the needle guide 500 or endoscope 1002, respectively. The pusher 1136 can be advanced using a mechanical advantage by use of a jackscrew, lever, or other threaded advance mechanism.

The endoscope 1002 comprises a catheter 1146 further comprising fiber-optic channels for optical viewing and for illumination of the target. The endoscope 1002 proximal end further comprises an eyepiece affixed to and operably connected to the endoscope hub 1120, which is optically and operationally connected to the fiber-optic channels running through the catheter 1146. The illumination port 1116 is affixed to the hub 1120 and is operably connected to fiber-optic channels that run through the length of the catheter 1146. The hub 1120 further comprises a deflecting lever 1118 which is affixed to pull-wires or pushrods which run from the deflecting lever 1118 to the distal end of the endoscope, where they are affixed to the catheter structure so as to provide a bending moment on the endoscope 1002 distal end. The hub 1110 is fabricated from polymers such as, but not limited to, polyethylene, polypropylene, polycarbonate, polyimide, polyurethane, polysulfone, and the like. The system is preferably provided sterile, having been packaged in a single or double pouch or tray arrangement, and then undergoing either ethylene oxide sterilization or gamma irradiation. The delivery system 300 can comprise radiopaque markers at or near the distal tip. The radiopaque markers are fabricated from materials such as, but not limited to, platinum, gold, tantalum, iridium, or a combination of the aforestated materials. Radiopacity can also be increased by vapor deposition coating or plating metal parts of the elongate tubular member 408 with metals or alloys of gold, platinum, tantalum, platinum-iridium, and other suitable materials. The radiopaque markers can be aligned so as to depict or convey an orientation, which is visible under X-ray or fluoroscopy. In another embodiment, the polymeric materials of the catheter or sheath may be loaded with radiopaque filler materials such as, but not limited to, bismuth salts, barium salts, or the like, at percentages ranging from 1% to 50% by weight in order to increase radiopacity. The radiopaque markers allow the delivery system to be guided and monitored using fluoroscopy.

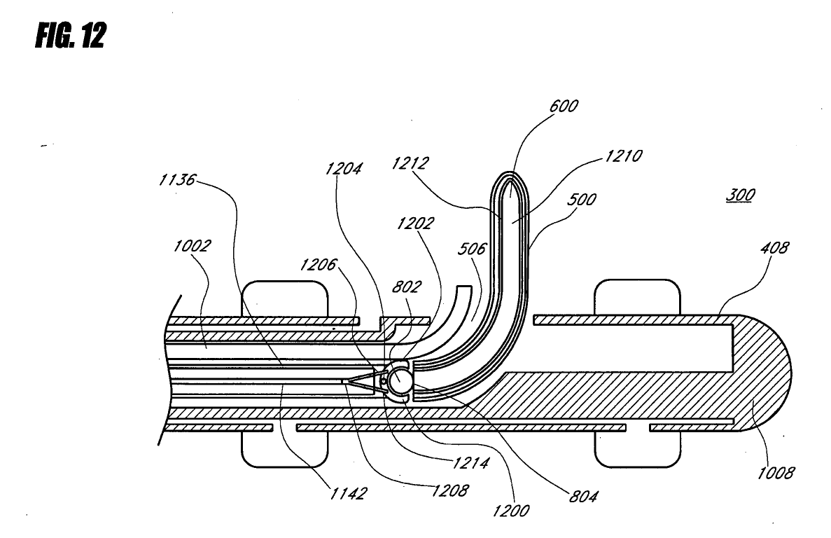

FIG. 12 illustrates a longitudinal cross-sectional view of the distal region of a delivery system 300 further comprising a pusher 1136, an implant 600, and a coupler 802 further having an undercut 804. The implant 600 further comprises an outer layer 1212 and an inner core 1210. The implant 600 and the needle guide 500 are shown in cross-section and bend out of the plane near the top of the view so that they appear to have an elliptical end but the distal end of the implant 600 and the needle guide 500 are not visible in this section. The delivery system 300 further comprises the elongate tubular member 408 and a distal tip 1008. The endoscope 1002 is shown in FIG. 12, as is the pusher 1136. The distal end of the pusher 1136 comprises an upper jaw 1202 and a lower jaw 1200 which are rotatably affixed to the distal end of the pusher 1136 by a pin or axle 1204. The coupler linkage 1142 is affixed to the upper jaw 1202 and the lower jaw 1200 by way of a connector 1208 and two sub linkages 1206 which are affixed to the upper and lower jaws 1202 and 1200 by the connections 1214.

Referring to FIG. 12, the pusher 1136 is advanced distally, relative to the needle guide 500, to deploy the implant 600 within tissue structures (not shown). Significant force can be required to advance the pusher 1136 and force the implant to bluntly dissect tissue. Such forces may be derived through application of mechanical, electrical, pneumatic, or hydraulic systems at the proximal end of the delivery system 300 and are transmitted through the delivery system 300 by the pusher 1136, which in certain embodiments may comprise a tube having significant column strength while still retaining flexibility. The pusher 1136 is retained in shape by the walls of the needle guide 500. Once the implant 600 is delivered to the correct location and its position is verified by fluoroscopy, endoscopy, MRI, ultrasound, and the like, the jaws 1202 and 1200 are retracted by pulling the coupler linkage 1142 proximally, which opens the jaws 1202 and 1200 so that the coupler 800 is released from the pusher 1136. The system allows for reattachment of the implant 600 to the pusher 1136, at least immediately after deployment and release. In other embodiments, the coupler could comprise a magnetic latch, a fusible link, an electrolytically erodeable link, a hydraulic expansion coupler, a friction coupler that is overcome by hydraulic or mechanical force, or the like. The force necessary to operate the coupler is transmitted through the delivery system by linkages, electrical cables, fluid lines or the like.

In an embodiment, a portion, or all of the implant 600 can comprise biodegradeable materials such as, but not limited to, sugars, polylactic acid, polyglycolic acid, collagen-based materials, combinations of these materials, and the like. Thus, the implant 600 can be made to materially dissolve in around 2 weeks to 104 weeks and preferably between 4 weeks and 52 weeks. In another embodiment, the implant 600 can comprise shape-memory polymers such as those described in U.S. Pat. Nos. 6,388,043 and 6,720,402, to Langer et al., the entirety of which are hereby incorporated herein by reference. In another embodiment, the implant 600 can comprise shape-memory polymers that are biodegradeable, biodissolvable, or bioerodable. In another embodiment, the implant core material 1210 can comprise metallic nitinol, a polymeric shape memory material or a simple spring metal such as stainless steel 304, cobalt nickel alloys, or the like. In the nitinol embodiment, the material is generally shape set so that upon exposure to a temperature in excess of the austenitic finish temperature, the material forms a circular shape which is smaller in diameter than its implant shape. The austenite finish temperature, in this embodiment, is preferably slightly higher than body temperature but can be between 30 and 50 degrees centigrade. The outer layer 1212 can be a separate tube, which is implanted first, and then the core material 1210 is inserted subsequently, potentially more than one time.

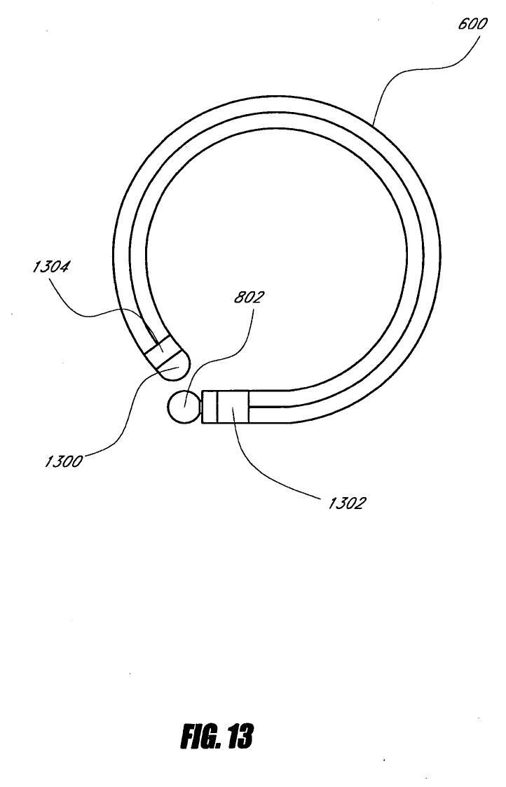

FIG. 13 illustrates an adjustable implant 600 comprising a blunt dissecting distal tip 1300 and a coupler 802. The blunt dissecting distal tip 1300 can be round or bulbous. In the illustrated embodiment, the blunt dissecting distal tip 1300 is elliptical in shape. The blunt tip 1300 is preferably not sharp and so cannot cut through tissue such as skin or other membranes or vessel walls. It can dissect planes through muscle and between muscle, ligaments, and fat when forcibly advanced distally. In the illustrated embodiment, the implant 600 is approximately circular in configuration. Referring to FIGS. 7 and 8, as the implant 600 is expelled through the needle guide 500, the implant 600 forcibly attempts to maintain a circular configuration and so takes a circular path once deployed. In another embodiment, the blunt tip 1300 comprises a slightly sharpened end to cut slightly, although the rounded edges serve as a standoff and prohibit the tip from cutting critical tissue such as the esophagus or aorta. In an embodiment, the implant 600 is wider lengthwise than it is radially thick. The width of the implant 600 can be between 0.5-mm and 30-mm, and preferably between 2 and 15-mm. The implant 600 thus has a tip that is complex in shape but appears as shown when viewing from along the axis of the major curvature. The implant 600 further can comprise radiopaque markers 1302 at its proximal end and radiopaque markers 1304 at its distal end as well as at an intermediate location (not shown). The implant 600 can further comprise permanent magnets that can be used to interact with an array of circumferentially arranged Hall-effect sensors on the delivery system (not shown) to determine the degree of circumferential deployment.

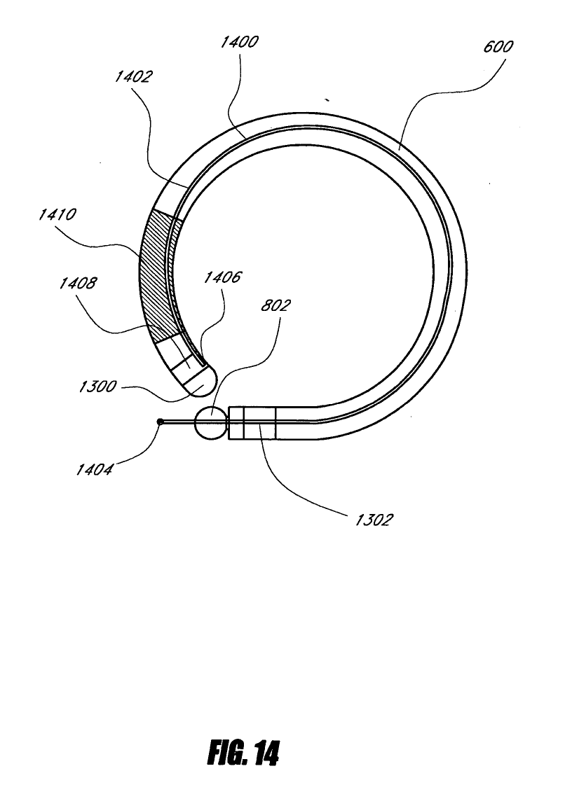

FIG. 14 illustrates a top cross-sectional view of an adjustable implant 600 comprising an internal steering mechanism. The implant 600 comprises the coupler 802, a pull-wire 1400, a pull-wire lumen 1402, a pull-wire connector 1404, a distal anchor 1408, a distal anchor connection 1406, and a flexible region 1410. The implant 600 is releasable from the delivery system by means of mechanisms similar to that shown in FIG. 12. In this embodiment, the coupler 802 comprises a through lumen and the pull-wire 1400 is slidably disposed therethrough. The pull-wire 1400 is slidably disposed within the pull-wire lumen 1402, which constrains the pull-wire 1400 from movement substantially away from the longitudinal axis of the pull-wire lumen 1402. The delivery system (not shown) comprises a separate pushrod (not shown) with openable jaws (not shown), similar to the jaws shown in FIG. 12 but the pull-wire coupler. Proximal withdrawal of the pushrod, in the delivery system, causes the pull-wire 1400 to undergo tension, which exerts tension on the off-center distal anchor connection 1406. This off-center tension causes the implant 600 to be coerced into a tighter radius. The flexible region 1410 aids the steering in that it selectively flexes more than the rest of the implant 600 and allows the distal end of the implant to curve inwardly more than if the flexible region 1410 was not present. The pull-wire 1400 could also be a pushrod affixed at the distal end such that compression of the pushrod would increase force on the outside of the implant 600 causing it to increasingly curve inward. Conversely, tension on the pushrod would cause the inward curve of the implant 600 to decrease. The motive power for the curving or articulation can also be obtained from actuators such as electrical motors or nitinol actuators.

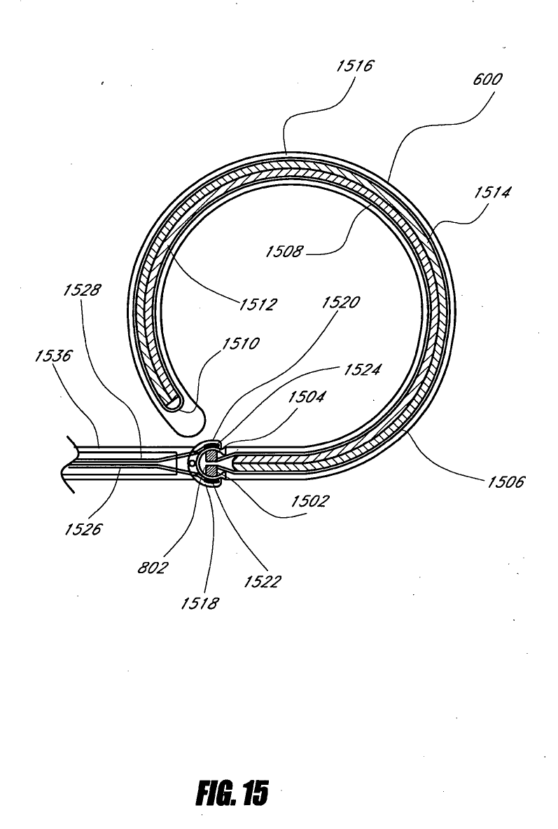

FIG. 15 illustrates a top view of an implant 600 comprising a releasable connection for electro-thermal adjustment of the implant 600. The implant 600 comprises a releasable coupler 802, a positive coupler electrode 1502, a negative coupler electrode 1504, a first length of heating element 1506, a second length of heating element 1508, a heating element shunt 1510, a first shape-memory element 1512, a second shape memory element 1514, and an outer encapsulating layer 1516. The releasable coupler 802 is affixed, or integrally formed, to the proximal end of the implant 600. The first and second heating elements 1506 and 1508 can be wires routed along the long axis of the implant 600, or helically routed as a coil along the long axis of the implant 600. The first and second heating elements 1506 and 1508 are preferably electrically insulated, on their exteriors, to prevent short-circuiting together at a point between the coupler 802 and the shunt 1510. The shunt 1510 is a wire that is affixed to and operably connects the distal ends of the heating elements 1506 and 1508. In an embodiment, the shunt 1510 can be integral to the heating elements 1506 and 1508, thus resulting in a single integral heating element.

The first shape memory element 1512 and the second shape memory element 1514 can be fabricated from nickel-titanium alloys. The shape memory elements 1512 and 1514 can be pre-set with different austenite finish temperatures. In an embodiment, the first shape memory element 1512 comprises material with a lower austenite finish temperature than that of the second shape memory element 1514. When electrical power is applied to the electrical connectors 1502 and 1504, the heating elements 1506 and 1508 raise the temperature of the shape memory elements 1512 and 1514 to a known, pre-calibrated temperature. The first shape memory element 1512 is pre-shaped to be biased toward a smaller diameter upon exposure to a temperature above the austenite finish temperature thus coercing the implant 600 into a smaller diameter. However, if an increased amount of electrical power is applied to the heating elements 1506 and 1508 the temperature rises to a level higher than the austenite finish temperature of the second shape memory element 1514. The second shape memory element 1514 is configured to expand its diameter upon exposure to temperatures higher than the austenite finish temperature. The second shape memory element 1514 can be configured to have a greater cross-section and a stronger resultant force that substantially overcomes, at least to some degree, the force applied by the first shape memory element 1512 and so it can bend the entire implant 600 outward to a larger diameter. In another embodiment, only a single shape memory element is used. In another embodiment, the first shape memory element 1512 expands the implant 600 and the second shape memory element 1514 contracts the implant 600. In another embodiment, shape memory polymers are comprised by the implant 600, rather than, or in addition to, nitinol. The outer coating 1516 can be a polymer such as, but not limited to, PTFE, polyester, polyethylene, polypropylene, silicone elastomer, or the like.

The electrical contacts 1502 and 1504 are configured to operably connect to electrical contacts 1522 and 1524, on the inside of the jaws 1518 and 1520 respectively, of the coupling mechanism on the distal end of the pusher 1136. Once the jaws 1518 and 1520 are closed around the coupler 802 and the electrical contacts are secure, electrically insulating material (not shown) can be coated over the entire assembly to prevent electrical losses. Electrical energy or power is supplied through the pusher 1136 by electrical lines or leads 1526 and 1528, which are electrically insulated or isolated from each other within the pusher 1136. The electrical lines or leads 1526 and 1528, in this embodiment, serve the additional function of providing mechanical traction or tension to open the haws 1518 and 1520 at the desired time. The jaws 1518 and 1520 can be keyed to fit over the coupler 802 in only certain orientations to ensure that electrical contact is made should re-attachment and adjustment be necessary. This configuration allows for adjustment of the implant 600 diameter at the time of initial placement. All exposed electrical contacts can be fabricated from stainless steel, platinum, gold, or the like so that they are biologically inert and can also have substantial radiopacity. The configuration also allows for potential adjustment of the implant at.a later date by re-connecting the electrical contacts 1502 and 1504 on the implant 600 to an electrical source (not shown). In another embodiment, the energy is delivered through the pusher 1136 by fluid lines (not shown) through which heated or refrigerating fluid is pumped. These fluid lines are operably connected to the heating elements 1506 and 1508, which are fluid carrying tubes in this embodiment. The heating elements 1506 and 1508 are operably connected by the shunt 1510 and can either heat or cool the shape memory elements 1506 and 1508.

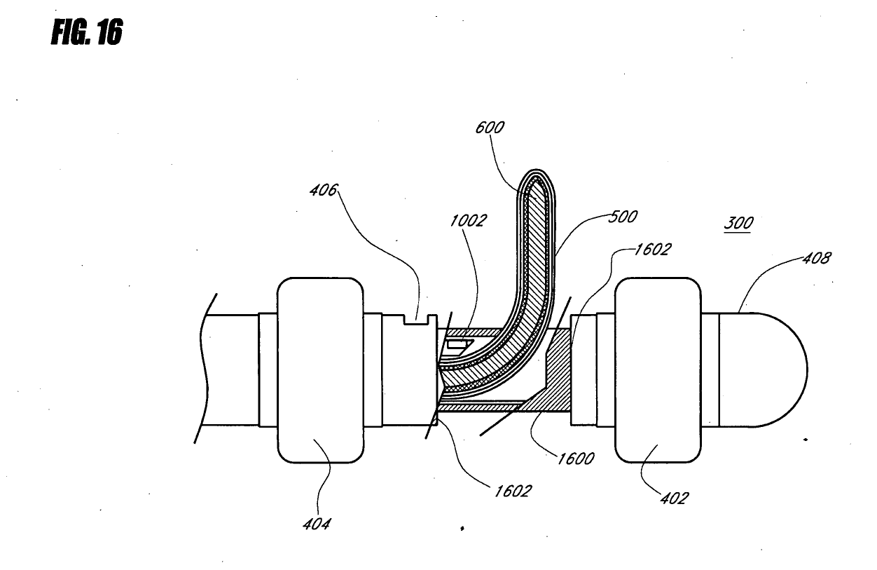

FIG. 16 illustrates a side view of the distal end of a delivery system 300 comprising a guiding groove 1600, according to an embodiment of the invention. The guiding groove 1600 is a circumferential depression in the delivery system tubing 408. The guiding groove 1600 further comprises the edges 1602 disposed at the distal and proximal end of the guiding groove 1600. The guiding groove 1600 serves to form a track in tissue, which is pulled down against the delivery system tubing by the vacuum exerted through the vacuum port 406 and maintained between the occlusion balloons 402 and 404. The needle guide, or guide sleeve, 500 penetrates the tissue in the region of the guiding groove 1600 and the implant 600 is extruded outward so as to follow the circumference of the body lumen or vessel, in this case an esophagus, within the depression or track formed in the tissue by the guiding groove 1600. The implant 600 is coerced against movement outside the track by the walls 1602 of the guiding groove 1600.

The delivery system 300 is used in conjunction with, and is operably connected to, a vacuum source, a light source, a video camera and monitor, a video recorder, a balloon inflation system, an irrigation system, an electrical heating source, and other equipment. The delivery system 300 is operably connected to this equipment at its proximal end through connectors, which can be Luers, Luer locks, CPC™ connectors, or other quick connectors. The system is provided sterile in single or double aseptic packaging and is sterilized using gamma irrigation, electron beam irradiation, ethylene oxide, or other suitable sterilization methodology.

In another embodiment, the degree of sphincter competence is assessed or measured in order to provide information on the degree of adjustment necessary in the implant 600. In this embodiment, the delivery system 300 is withdrawn partly, leaving electrical connections in place between an external power source and the implant 600. A small catheter can be extended into the stomach through the lower esophageal sphincter and the stomach filled with fluid such as water or air. The degree of sphincteric incompetence can be observed using an endoscope or other sensor and adjustments can be made in the implant diameter to generate optimal sphincter function. At this time, the electrical connections to the implant can be detached and the entire delivery system, catheter, endoscope, and other equipment withdrawn from the patient.

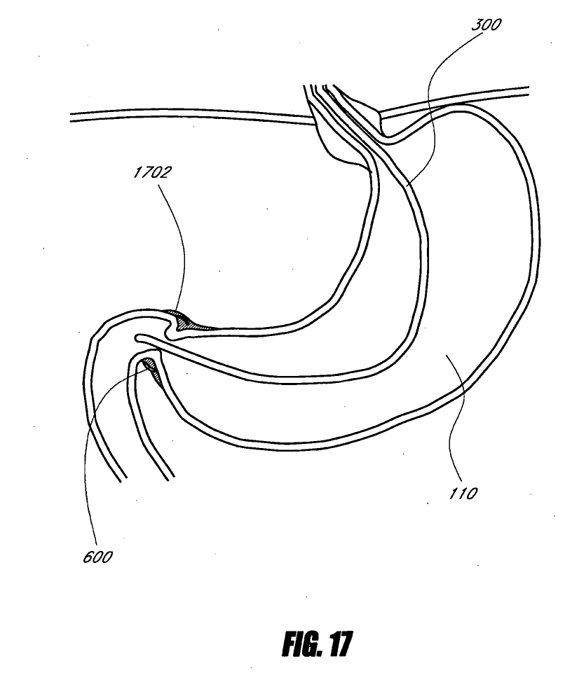

FIG. 17 illustrates a cross-sectional view of the stomach 110 as viewed from the anterior side and looking posteriorly. A delivery system 300 has been placed transesophageally into the stomach 110 and routed within the lumen surrounded by the pylorus muscle 1702. An implant 600 has been deployed and detached from the delivery system 300 and the guide sleeve (not shown) has been retracted within the delivery system 300. In this embodiment, the implant 600 is capable of correcting or modifying the closure of the pyloric sphincter, which is located near the distal end of the stomach 110 between the stomach and the duodenum, which is the proximal part of the small intestine. The implant is embedded, at least partially, within the pylorus muscle 1702. Such placement is capable of controlling the rate of stomach emptying as well as having an effect on the competence of the pyloric sphincter 1702.

The present invention may be embodied in other specific forms without departing from its spirit or essential characteristics. For example, the delivery system can include instruments affixed integrally to the interior central lumen of the sheath, rather than being separately inserted, for performing therapeutic or diagnostic functions. The hub may comprise tie downs or configuration changes to permit attaching the hub to the mouth or face of the patient. The system can be used in the stomach to create constrictions or bands to compress the stomach and restrict the flow of nutrients into or through the stomach. Various valve or seal configurations and radiopaque marker configurations are appropriate for use in both the delivery system and the implant. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

What is claimed is:

1. A delivery system, for placing an implant at least partially outside a body lumen, comprising:
an elongate member having a sidewall, distal and proximal ends, and a lumen extending through the elongate member;
a piercing guide axially slidable within said lumen of said elongate member, said piercing guide configured to extend radially outwardly from, or retract radially inwardly into, an aperture in the elongate member at or near the elongate member's distal end, said piercing guide having (1) a sharp distal end, configured to penetrate tissue surrounding the body lumen, and (2) a lumen extending through the piercing guide;
a pusher configured to move axially an elongate implant within said piercing guide lumen, wherein said axial movement is controllable by a control mechanism at or near the proximal end of the delivery system;
a coupler at a distal end of the pusher, said coupler configured to couple releasably the implant to the pusher, wherein release of the implant is controlled by a release mechanism located at or near the proximal end of the delivery system.

2. The apparatus of claim 1, further comprising:
a first expandable member on or in the distal end of the elongate member, distal of said aperture, said first expandable member configured to be inflated upon introduction of an inflation fluid into the interior of said first expandable member; and
an inflation lumen having distal and proximal ends extending though said elongate member, said inflation lumen having at least one port operably positioned for communicating with the interior of said first expandable member.

3. The apparatus of claim 2, further comprising:
a second expandable member on or in the elongate member, proximal to said first expandable member and said piercing guide aperture, said second expandable member configured to be inflated upon introduction of an inflation fluid into the interior of said second expandable member;
wherein an inflation lumen in said elongate member comprises a second port operably positioned for communicating with the interior of said second expandable member.

4. The apparatus of claim 1, further comprising:
a first expandable member on or in the distal end of the elongate member, distal of said aperture, said first expandable member configured to be expand upon actuation and to engage tissue around said body lumen.

5. The apparatus of claim 4, further comprising:
a second expandable member on or in the elongate member, proximal to said first expandable member and said piercing guide aperture, said second expandable member configured to be expand upon actuation and to engage tissue around said body lumen.

6. The apparatus of claim 5, further comprising:
at least one vacuum port located in the sidewall of said elongate member between said first and second expandable members;
a vacuum lumen having distal and proximal ends extending though said elongate member, the distal end of said vacuum lumen in fluid communication with said vacuum port; and
a vacuum connector located at the proximal end of the delivery system, said vacuum connector in fluid communication with said vacuum lumen, wherein application of negative pressure to the vacuum connector creates negative pressure in a space between the first and second expandable members and between the sidewall of the elongate member and an inner surface of the body lumen.

7. The apparatus of claim 1, wherein the piercing guide is configured to be move radially outwardly or inwardly through the aperture in a direction substantially perpendicular to the longitudinal axis of the elongate member.

8. The apparatus of claim 1, wherein a distal tip of said piercing guide is beveled between 20-70° with respect to the longitudinal axis of the piercing guide.

9. The apparatus of claim 1, wherein the piercing guide comprises a shape memory material.

10. The apparatus of claim 1, wherein the piercing guide is configured such that a distal tip may be aligned substantially tangential to the circumference of a wall of the body lumen, following penetration of said body lumen.

11. The apparatus of claim 10, further comprising a control mechanism configured to articulate a distal end of the piercing guide.

12. The apparatus of claim 1, further comprising an elongate implant, said implant having distal and proximal ends and a blunt distal tip, the implant having a first, implant shape and a second, delivery shape, wherein the implant is configured to be positioned in said piercing guide lumen in said second, delivery shape and to transform to said first, implant shape upon or after advancement from said piercing guide lumen.

13. The apparatus of claim 1, further comprising a closure device configured to assist in closing an opening in said tissue created upon said penetration of said tissue.

14. The apparatus of claim 1, further comprising an endoscope slidably positioned within said elongate member.

15. A method, of placing of an implant within a portion of a mammalian gut, comprising:
at least partially puncturing a mammalian gut wall, such that at an opening in at least part of the gut wall is created, said opening extending between a first layer and a second layer of tissue within the gut wall;
inserting an implant comprising a shape memory material into said opening in the gut wall, said implant having a first delivery configuration and a second configuration;
advancing said implant through said gut wall, between said first and second layers of tissue;
closing the opening in the gut wall such that the implant is wholly retained between said tissue layers.

16. The method of claim 15, further comprising activating the shape memory material to transform said implant from said first configuration to said second configuration.

17. The method of claim 16, wherein said activating comprises applying an activation energy from within a lumen of said gut, proximal to said implant.

18. The method of claim 16, wherein said activating comprises activating said shape memory material from outside a patient's body.

19. The method of claim 15, further comprising adjusting a diameter of the implant.

20. The method of claim 19, further comprising adjusting a diameter of the implant after the implantation procedure.

21. The method of claim 19, wherein adjusting the diameter comprises applying energy to raise the temperature of the implant causing a shape-memory reaction to occur in the implant.

22. The method of claim 19, wherein adjusting the diameter comprises:
removing energy from the implant to reduce the temperature of the implant; and
expanding the implant to a larger diameter.

23. The method of claim 15, wherein advancing the implant further comprises bluntly dissecting the gut wall tissue between the first and second layers.

24. The method of claim 15, further comprising:
providing a delivery system, comprising: (1) an elongate member having distal and proximal ends, and having a delivery lumen extending therebetween; and (2) a guide sleeve slidably inserted in a lumen of said elongate member;
advancing the delivery system to or near said portion of the gut;
advancing said guide sleeve radially outward from an aperture in the elongate member; and
puncturing a wall of said gut with a distal tip of the guide sleeve.

25. The method of claim 24, further comprising:
positioning said delivery system such that the portion of gut is located between a distal and a proximal expandable member mounted at or near the distal end of the elongate member;
inflating the distal expandable member;
inflating the proximal expandable member; and
drawing a vacuum in the region between the proximal and the distal expandable members to pull the portion of mammalian gut toward the elongate member.

26. The method of claim 25, wherein advancing said implant further comprises advancing said implant through said guide sleeve.

27. The method of claim 15, wherein said portion of gut wall comprises a sphincter.

28. The method of claim 15, wherein said portion of gut wall comprises a portion of a stomach.

29. The method of claim 15, wherein said portion of gut wall comprises a portion of an esophagus.

30. The method of claim 15, wherein said portion of gut wall comprises a portion of a colon.

31. The method of claim 15, wherein advancing said implant further comprises tunneling said implant within said gut wall.

32. A method of placing of an implant around a portion of a mammal's gut comprising:
puncturing a portion of the gut wall, such that an opening in the gut wall is created, said opening extending from within a lumen of the gut through the gut wall;
inserting an implant having a first delivery configuration and a second configuration through said opening in the gut wall, said implant;
placing said implant near an outer circumference of said gut; and
closing the opening in the gut wall such that the implant at least partially surrounds said gut and resides between the gut wall and the mammal's visceral peritoneum.

33. The method of claim 32, further comprising transforming said implant from said first configuration to said second configuration.

34. The method of claim 33, wherein said implant comprises a shape memory material, and said transforming further comprises activating the shape memory material.

35. The method of claim 33, wherein the implant comprises an adjustable steering mechanism and wherein transforming said implant comprises electrically actuating the implant.

36. An implant for adjusting a diameter of a portion of a mammalian gut, comprising:
an outer sheath having a proximal end and a distal end, wherein the outer sheath is configured to assume a first, elongate shape when constrained and to transform to a second, substantially circular shape when unconstrained;
a blunt dissecting tip located on the distal end of the outer sheath;
a coupler located at the proximal end of the outer sheath, wherein the coupler is configured to couple releasably to a delivery system pusher; and
an inner core comprising a shape memory material configured to adjust a diameter of the implant when the implant is in said second, unconstrained configuration and said shape memory material is activated.

37. The implant of claim 36, wherein the inner core comprises at least two different shape-memory elements, wherein each shape memory element has a different transition temperature from another shape memory element.

38. The implant of claim 36, wherein the outer sheath is configured to adjust the diameter of the implant.

39. The implant of claim 36, wherein the outer sheath comprises at least in part a shape memory material.

40. The implant of claim 36, wherein the outer sleeve comprises a biodegradeable material.

41. The implant of claim 36, wherein the inner core comprises a biodegradeable material.

42. The implant of claim 36, wherein the implant comprises an outer cross-section with a substantially flattened shape.

43. An implant for adjusting a diameter of a portion of a mammalian gut, comprising:
an outer sheath having a proximal end and a distal end, wherein the outer sheath is configured to assume a first, elongate shape when constrained and to transform to a second, substantially curvilinear shape when unconstrained;
a blunt dissecting tip located on the distal end of the outer sheath;
a coupler located at the proximal end of the outer sheath, wherein the coupler is configured to couple releasably to a delivery system pusher; and
an inner core comprising a diameter-changing member having distal and proximal ends, said distal end coupled to the distal end of said outer sheath, wherein movement of said diameter-changing member relative to said outer sheath causes a diameter of said implant to change.

44. The implant of claim 43, further comprising an electrical connection at or near the coupler, wherein said electrical connection is configured to actuate electrically said diameter changing member upon application of energy.

45. The implant of claim 43, wherein the diameter-changing member comprises a shape-memory material.
